(19) United States
(12) Patent Application Publication
(10) Pub. No.: US 2012/0016441 A1
(43) Pub. Date: Jan. 19, 2012

(54) DEVICE FOR ELECTRIC PULSE STIMULATION OF HEALING OF WOUNDS

(76) Inventors: Sergei Belov, Moscow (RU); Anna Pshelenskaj, Moscow (RU); Viktor Salyuk, Moscow (RU); Oleg Sushkov, Moscow (RU); Yurii Danileiko, Troitsk (RU); Sergei Nefedov, Moscow (RU); Viacheslav Osiko, Moscow (RU); Sergei Frolov, Moscow (RU)

(21) Appl. No.: 12/839,195

(22) Filed: Jul. 19, 2010

(51) Int. Cl. A61N 1/00

(52) U.S. Cl. 607/50

(57) ABSTRACT

A device that improves the efficiency of the treatment process, shortens healing time, and treats large-area wound surfaces and various forms of local inflammatory processes comprising an HF generator, power source, timer, pedal, electronic module control device, and a device for delivering exposure energy from the generator to biological tissue, wherein the pedal is pressed, a signal arrives at a timer and electronic module for controlling a device for the metered movement of a needle electrode. By means of this module, movement to a specified depth can be ensured. After the needle electrode is inserted into biological tissue, a signal to turn on the HF generator is sent automatically from the timer and a movement sensor. The current of the HF generator is modulated by a modulator that is fed to needle electrode. The timer turns off the HF generator, and the needle electrode is extracted from the biological tissue.

### CROSS-REFERENCE TO RELATED APPLICATION

Not Applicable

### FEDERALLY SPONSORED RESEARCH

Not Applicable SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention pertains to medical technology and can be used for treating traumatic and postoperative wounds, including infected wounds, as well as various forms of local inflammatory processes.

Known is a multichannel device (“The Electropuncture Device” SU (11) 525961 (13) A1) used for the treatment of inflammatory processes using the electropuncture method. The device comprises an electric voltage generator, a power supply module and an electric voltage switching module connected to needle-type working electrodes. The device's shortcoming is its low efficiency in the treatment process and considerable length of treatment time due to the repetitive nature of the treatment procedure.

Known is a device for the surgical treatment of the inflammatory processes of pilonidal fistula [patent RU (11) 2283632 (13) C1], which is the prototype of the proposed device. The device comprises a semiconductor laser, a power supply and control module, a timer, a pedal, and a system for light-guide delivery of laser radiation to the exposure area. The semiconductor laser operates in continuous mode with a radiation wavelength of 970 nm and power up to 2.5 W. Operation of the device involves laser treatment of the cavity of pockets and fistulous tracts, with purification and evaporation of devitalized and changed biological tissues. The treatment is performed using a two- or three-stage intrastitial laser thermotherapy. A flexible fiber light guide is used as the means for delivery of laser radiation to exposure area; its distal end is inserted in the fistulous tract through primary holes, or transcutaneously. The depth of insertion of the light guide distal end is controlled by means of ultrasound. The length of laser exposure is in the range of 30-180 s.

The device's shortcomings are the complexity of treating a large area of the wound surface; the need to conduct a large number of radiation sessions; a patient's individual sensitivity to the procedure; and the difficulty of optimizing the radiation dose in terms of time, area and depth of impact for a given patient.

All these shortcomings reduce the treatment process's efficiency and increase the healing time of wounds during the postoperative period.

The object of the present invention is to develop a device that improves the efficiency of the treatment process, shortens the healing time, and provides for the ability to treat large-area wound surfaces and various forms of local inflammatory processes (for instance, healing of wounds of the perianal and sacrococcygeal region in the postoperative period for patients operated for perianal fistulas with various localizations, as well as chronic inflammation of the pilonidal fistula, pyoderma, etc.).

### SUMMARY

The object of the present invention is to develop a device that improves the efficiency of the treatment process, shortens the healing time, and provides for the ability to treat large-area wound surfaces and various forms of local inflammatory processes (for instance, healing of wounds of the perianal and sacrococcygeal region in the postoperative period for patients operated for perianal fistulas with various localizations, as well as chronic inflammation of the pilonidal fistula, pyoderma, etc.).

The invention is a device for electric pulse stimulation of the healing of wounds and local inflammatory processes comprising a generator, a power source, a timer, a pedal, an electronic module control device, and a device for delivering exposure energy from the generator to biological tissue, distinctive in that, in order to improve the efficiency of the treatment process, shorten the healing time, speed up treatment of large-area wound surfaces, as well as of various forms of inflammatory processes (for instance, healing wounds of the perianal and sacrococcygeal region during the postoperative period for patients operated for rectal fistulas with various localization, as well as chronic inflammations of pilonidal fistula, pyoderma, etc.), a high frequency (HF) generator is used as the generator. The invention further comprises an HF voltage modulator, an instrument for high frequency current action on biological tissue in the form of a set of needle electrodes, an electromechanical module for controlling a device for the metered movement of the needle electrodes, and a needle electrodes movement sensor.

The device works as follows. When a pedal is pressed, a signal arrives at a timer. From the timer, the signal arrives at an electronic (or electromechanical) module for controlling a device for the metered movement of a needle electrode; by means of the electronic (or electromechanical) module for controlling the device for the metered movement of the needle electrode, movement to a specified depth can be ensured. After the needle electrode is inserted into biological tissue, a signal to turn on an HF generator is sent automatically from the timer and a movement sensor; the current of the HF generator is modulated by a modulator that is fed to needle electrode. After the HF current set exposure time (1-10 s), the timer turns off the HF generator, and the needle electrode is automatically extracted from the biological tissue.

### DRAWINGS

FIG. 1 comprises a device for the realization of the proposed method, comprising an HF voltage generator, a modulator providing the required relative duration of high frequency voltage pulses, a timer, a pedal, a device for the metered movement of a needle electrode with a movement sensor, and an electronic module for controlling the metered movement of the needle electrode.

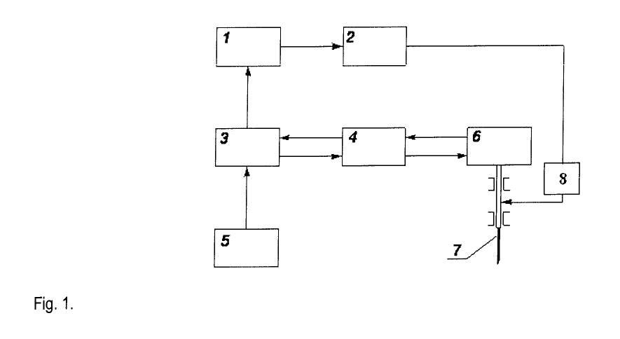

### REFERENCES

1—HF generator

2—modulator

3—timer

4—electronic (or electromechanical) module for controlling the device for the metered movement of the needle electrode

5—pedal

6—device for the metered movement of the needle electrode

7—needle electrode

8—movement sensor

### DESCRIPTION

The device works as follows. When a pedal 5 is pressed, a signal arrives at a timer 3. From the timer 3, the signal arrives at an electronic (or electromechanical) module 4 for controlling a device 6 for the metered movement of a needle electrode 7; by means of the electronic (or electromechanical) module 4 for controlling the device 6 for the metered movement of the needle electrode 7, movement to a specified depth can be ensured. After the needle electrode 7 is inserted into biological tissue, a signal to turn on an HF generator 1 is sent automatically from the timer 3 and a movement sensor 8; the current of the HF generator 1 is modulated by a modulator 2 that is fed to needle electrode 7. After the HF current set exposure time (1-10 s), the timer turns off the HF generator, and the needle electrode is automatically extracted from the biological tissue.

The HF generator 1 parameters are as follows: Operating frequency 100 kHz-24 MHz; Modulation frequency 0.5-10 kHz; Filling factor 1-30%; and Output voltage amplitude 120-300 V.

The parameters of the device 6 of the electromechanical module 4 for the metered movement of the needle electrode 7 are as follows: the needle electrode linear movement range is 0-15 mm; and the needle electrode movement (insertion-extraction) speed range is 0.1-2 cm/s.

The proposed device can operate in two independent modes.

First Operating Mode: The electronic (or electromechanical) module 4 for the metered movement of the needle electrode is placed on the wound surface. The HF generator's 1 operating frequency is set in the 1-24 MHz range, the filling factor is set in the 1-30% range, the pulse frequency is set in the 0.5-10 kHz range, the HF current exposure time is set in the 3-10 seconds range, and the voltage amplitude is set in the 120-200 V range. The pedal 5 is pressed, and the signal from the pedal 5 arrives at the timer 3. From the timer 3, the signal arrives at the electronic (or electromechanical) module 4 for controlling the metered movement of the needle electrode 7; from the electronic (or electromechanical) module 4, a signal to turn on the electronic (or electromechanical) module for the metered movement of the needle electrode 7 is sent. The movement of the needle electrode 7 begins; it is inserted in biological tissue perpendicularly to the wound surface to the preset depth of 1 mm to 15 mm. After the needle electrode 7 is inserted in the biological tissue, a signal to turn on the HF generator 1 is sent automatically from the timer 3; the HF generator 1 current, modulated by the electronic (or electromechanical) modulator 4, is fed to the needle electrode 7. After the HF current set exposure time (1-10 s), the timer 3 turns on the HF generator 1, and the needle electrode 7 is automatically extracted from the biological tissue. Then the electronic (or electromechanical) module for the metered movement of the needle electrode 7 is moved to the new place, and the above procedure is repeated until the wound surface is treated completely.

Second Operating Mode: The electronic (or electromechanical) module 4 for the metered movement of the needle electrode 7 is placed on the wound surface. The filling factor is set in the 1-10% range, the pulse frequency is set in the 0.5-2 kHz range, and the voltage amplitude is set in the 200-300 V range. The pedal 5 is pressed, and a signal from the pedal 5 arrives at the electronic (or electromechanical) module 4 for controlling the metered movement of the needle electrode 7; from the electronic (or electromechanical) module 4, a signal to turn on the electronic (or electromechanical) module for the metered movement of the needle electrode 7 is sent. The movement of the needle electrode 7 begins; it is inserted in biological tissue perpendicularly to the wound surface to a preset depth from 4 mm to 15 mm. After the needle electrode 7 is inserted to the set depth, the timer 3 is turned on, and an HF voltage is applied to the needle electrode 7, while at the same time automatic extraction of the needle electrode from biological tissue begins without turning off the HF voltage. The speed of needle electrode 7 extraction from biological tissue is set in the 0.1-1.5 cm/s range. In this operating mode, the needle electrode 7 is a metal needle, 0.7 mm in diameter, coated with insulating material, with a 1-2 mm long distal working section. The HF voltage is turned off automatically by a signal from a movement sensor 8 when the 1-2 mm depth of insertion in the biological tissue is reached. Then the electronic (or electromechanical) module 4 for the metered movement of the needle electrode 7 is moved to a new place, and the above procedure is repeated until the wound surface is treated completely. In this case, the claimed device performs in a plasma mode for impacting biological tissue.

To expedite wound surface treatment, the module (applicator) for the needle electrodes can have up to 9 needles, with HF voltage applied to each of them alternately or simultaneously. Needle electrodes are extended from the device of the electromechanical module for the metered movement of needle electrodes independently of each other, either individually or in groups (set by the operating user on the electronic control module).

Such design of a multi-needle applicator makes it possible to substantially reduce the time of performing the electrical therapy procedure.

Clinical Example. Female patient A., 47 years old, clinical record No. 482-09, was admitted with complaints of purulent discharge from the anus and anal canal pain during defecation.

An external inspection of the perianal area revealed the following: the anus is closed, perianal reflex is active. In the six-o-clock area, a funnel-like retraction—a fistulous opening up to 2 mm in diameter—is detected. A digital investigation of rectum revealed the following: the sphincter tone and conation are satisfactory. In the six-o-clock area, a scar-changed fistulous opening up to 2 mm in diameter in the rear crypt area is detected. An examination with a bulbous-end probe revealed a trans-sphincter fistula tract.

The patient's diagnosis: rear trans-sphincter perianal fistula.

On Dec. 12, 2009, operative intervention was performed on the patient, namely, fistulectomy, and an opening and draining of an intersphincter leak. Two days after surgery, the procedure of electric pulse stimulation of the wound using the proposed method was administered to the patient. A multi-needle electrode with two needles was used. The HF current frequency was 2.64 MHz, the relative pulse-duty factor of 8, the voltage was 160 V, average power was 5 W, and the exposure time of each prick was 6 seconds. Using this method, 5 cm² of the surface was treated.

The postoperative period was without complications. During the 3^{rd} day after the procedure, appearance of marginal epithelization was observed; by the 9^{th} day after the operation, wound purification with complete formation of granulation tissue was observed; by the 14^{th} day, a reduction of the dimensions of the postoperative wound was observed; and on the 20^{th} day, complete epithelization of the wound with formation of a postoperative scar was determined macroscopically.

As a rule, the procedure is prescribed once, on the 2^{nd} day after operative intervention. In complex cases the procedure is repeated up to 3 times with a 3-4 day interval.

Thus, compared to known devices, the proposed device makes it possible to:
cut the time of wounds healing after surgery for rectal fistulas and chronic inflammation of pilonidal fistula due to acceleration and simultaneous running of the wound process phase;
reduce the number of postoperative bed-days; and
reduce the length of social and vocational rehabilitation.

1. A device for electric pulse stimulation of the healing of wounds and local inflammatory processes comprising a generator, a power source, a timer, a pedal, an electronic module control device, and a device for delivering exposure energy from the generator to biological tissue, distinctive in that, in order to improve the efficiency of the treatment process, shorten the healing time, speed up treatment of large-area wound surfaces, as well as of various forms of inflammatory processes (for instance, healing wounds of the perianal and sacrococcygeal region during the postoperative period for patients operated for rectal fistulas with various localization, as well as chronic inflammations of pilonidal fistula, pyoderma, etc.), a high frequency (HF) generator is used as the generator.

2. The device per claim 1, distinctive in that it contains an HF voltage modulator.

3. The device per claim 1, distinctive in that it contains an instrument for high frequency current action on biological tissue in the form of a set of needle electrodes.

4. The device per claim 1, distinctive in that it contains an electromechanical module for controlling a device for the metered movement of the needle electrodes.

5. The device per claim 4, distinctive in that it contains a needle electrodes movement sensor.

6. The device per claim 1, distinctive in that the HF generator generates HF voltage with a frequency from kHz to 24 MHz.

7. The device per claim 1, distinctive in the HF generator generates HF voltage with an amplitude from 120 V to 300 V.

8. The device per claim 2, distinctive in that the modulator modulates HF voltage with frequency from 0.5 kHz to 10 kHz.

9. The device per claim 2, distinctive in that the HF voltage modulator modulates HF voltage with a filling ratio from 1% to 30%.

10. The device per claim 3, distinctive in that the diameter of the needle electrodes is up to 0.7 mm.

11. The device per claim 3, distinctive in that the length of the needle electrodes is up to 15 mm.

12. The device per claim 4, distinctive in that it provides linear movement of the needle electrodes in the 0-15 mm range.

13. The device per claim 4, distinctive in that it provides the speed of the needle electrode linear movement in the range of 0.1 cm/s to 1.5 cm/s.

14. The device per claim 5, distinctive in that it provides for an automatic turning off of HF voltage to the needle electrodes by a signal from the movement sensor when the 1-2 mm depth of the needle electrodes insertion in biological tissue is reached.

15. The device per claim 1, distinctive in that it realizes two modes of biological tissue exposure that have different amplitudes of HF voltage applied to the needle electrodes.

16. The device per claim 15, distinctive in that in a first mode, the HF voltage amplitude is in the range of 120 V to 200 V.

17. The device per claim 16, distinctive in that the needle electrodes have no insulating coating.

18. The device per claim 16, distinctive in that HF voltage is applied to the needle electrodes after they are inserted in biological tissue to the 1-2 mm depth.

19. The device per claim 16, distinctive in that the turning off of HF voltage to the needle electrodes occurs after a time interval set on the timer.

20. The device per claim 16, distinctive in that extraction of the needle electrodes from biological tissue occurs automatically after turning off the HF voltage applied to them.

21. The device per claim 15, distinctive in that in a second mode, the HF voltage amplitude is in the range of 200 V to 300 V.

22. The device per claim 21, distinctive in that the needle electrodes have insulating coating except for their distal working section.

23. The device per claim 22, distinctive in that the needle electrodes have a 1-2 mm long uninsulated distal section.

24. The device per claim 21, distinctive in that application of HF voltage to the needle electrodes begins from the moment the stage of extracting them from biological tissue begins.

25. The device per claim 3, distinctive in that the needle electrodes module can comprise up to 9 needles.

26. The device per claim 4, distinctive in that the needle electrodes are extended from the electromechanical module that controls the device for the metered movement of the needle electrodes independently of each other.
